(19) United States
(12) Patent Application Publication
(10) Pub. No.: US 2016/0106343 A1
(43) Pub. Date: Apr. 21, 2016

(54) BREATH ANALYSIS SYSTEMS AND METHODS FOR SCREENING INFECTIOUS DISEASES

(71) Applicant: Capnia, Inc., Redwood City, CA (US)

(72) Inventors: Anthony D. WONDKA, San Ramon, CA (US); Anish BHATNAGAR, Redwood City, CA (US)

(21) Appl. No.: 14/918,484

(22) Filed: Oct. 20, 2015

Related U.S. Application Data
(60) Provisional application No. 62/066,094, filed on Oct. 20, 2014.

(51) Int. Cl. A61B 5/097; A61B 5/00; A61B 5/08

(52) U.S. Cl. CPC A61B 5/097; A61B 5/082; A61B 5/0816; A61B 5/749; A61B 2560/0443

(57) ABSTRACT

Breath analysis systems and methods test for infectious diseases in exhaled breath gas or condensate. An automatic breath sampling system can obtain reliable samples over a variety of clinical situations. In some variations, the system is modular system and can protect the equipment and clinician from contamination. In some variations, the system can be a point-of-care rapid-result instrument. In some variations, can be configured for off-line analysis in which case the collected sample is presented to a stand-alone analyzer for measurement.

### CROSS-REFERENCE TO RELATED APPLICATION

This application claims the benefit of U.S. Provisional Application No. 62/066,094, filed Oct. 20, 2014, the content of which is incorporated herein in its entirety.

### FIELD

This disclosure generally relates to the field of breath analysis and, more particularly, to screening, monitoring, diagnosing, and assessing the presence and status of an infectious disease in the breath.

### BACKGROUND

Screening for infectious diseases typically requires a blood test, or testing of another bodily fluid such as saliva, which can take several hours to perform, and usually needs to be performed at a central location where the blood analysis equipment is located. Blood or body fluid analysis is therefore not ideal for screening people for infectious diseases, unless the person is quarantined while the analysis is being performed, which is not practical and ineffective from a public health standpoint. In addition, screening people using blood, saliva or body fluid tests potentially exposes the health care workers involved in sample collection and analysis to the infectious agent, because of the patient contact and specimen handling required throughout the process.

Traditional screening techniques also do not lend themselves to individuals performing the screening test on themselves—a heath care worker is required to collect, handle, transport and analyze the sample. There is a large unmet need for a better screening procedure, especially during outbreaks and pandemics.

### BRIEF SUMMARY

An improved may include some of the following improvements: (1) it would lend itself to a lay person performing the test on him or herself, (2) it would be capable of a point-of-care rapid result, or optionally a rapid off-line analysis of the sample, (3) it would include a sample handling method that avoids cross contamination of test subjects and protection against infection of a health care worker, and (4) it would lend itself to convenient repeated use, as in the case of screening large crowds of people or repeating the test on singular subjects.

An improved screening method may involve testing for an infectious disease by measuring an analyte in the breath of the subject. For example, some viral infections, such as Ebola, may cause an elevated level of hemolysis, which can be detected by measuring CO in the breath. However, the level of CO may be present in trace amounts, which may be require high levels of precision and accuracy, and technically sophisticated measurement systems. In addition, special precautions need to be taken into account with the equipment to prevent cross contamination between patients and protect the health care provider from being exposed to the contaminant and contracting the disease.

Some infectious diseases may be detected in the breath condensate, such as proteins, bacteria, viruses and other solid molecules that are correlated to the presence of the disease. Other infectious diseases may be detected in the breath gas, by measuring a gaseous substance that is correlated to the presence of the disease. The analyte, whether in the condensate of the breath or in the breath gas, may be uniformly present in the breath, or may be located in a certain section of the lung, such as in the alveolar section. A breath analysis system may take this into account when performing the analysis.

In some instances, a test subject may not be willing to or not able to follow commands. If a certain type of breath maneuver is required to obtain the analysis, a reliable sample may not be able to be collected, or a sample may not be collectable at all, or, lead to inaccurate or false results, and therefore limited in its effectiveness. An effective infectious disease screening tool may need to be 100% accurate and 100% effective to be useful in this application. Therefore, an automatic breath collection and sampling system is highly preferred in a system that screens for infectious disease.

In some variations, the systems and method described herein are capable of solving one of more the existing problems with infectious disease screening. These systems and methods may employ an automatic and rapid point of care breath collection and analysis system for measuring for the presence of infectious disease markers, and may incorporate features to protect subjects from cross contamination and the health care workers from contracting the disease. The system is configured to be useful in field applications such as villages or clinics in remote areas or in battlefields, in public or semi-public areas such as transportation terminals or shelters, in health care settings such as emergency rooms and triage locations, and in the home. In the latter case, the system can be self-used by a subject at home that is screening themselves for exposure to a disease or for the progression or improvement of symptoms once infected.

While the examples given are related to infectious disease, the systems and methods described herein may also apply to poisonous agents. In some variations, multiple measurements are performed on an individual to assess the effectiveness of treatment and/or to help titrate and modulate the dose of the treatment. For example, if an antiviral agent or a blood transfusion or blood filtration therapy is applied to an affected individual, the effectiveness and status of the treatment can be monitored, as well as the dose of treatment. And while the examples given may relate to screening populations of people for outbreaks or unwanted exposure, the disclosure is not so limited and may apply to, for example, research applications in which the test subjects are intentionally exposed to the infection or toxin.

In some variations, a breath analysis apparatus includes: an inlet to obtain a flow of gas from a subject; a breath detector to measure a breathing signal in the flow of gas; a processor that determines an acceptable breath based on the breathing signal; a modular subassembly containing the pathway for the flow of gas that is removable from the apparatus; valves for controlling the flow of gas within the pathway, wherein the valves are fluidly disconnected from the flow of gas; and an analyte composition sensor fluidly connected to the flow of gas.

In some variations, the valves are configured to allow the removable gas flow pathway to be snapped into the apparatus. In some variations, the valves are pincher valves.

In some variations, the apparatus includes a peristaltic pump, and the breath detector includes a non-contact sensing element.

In some variations, the apparatus includes a transmitter to transmit results to a physically remote receive for viewing by a user at a distance from the apparatus. In some variations, the apparatus includes a receiver, and wherein the apparatus is configured to analyze breaths in response to remote commands received through the receiver from a physically separate transmitter.

In some variations, the apparatus includes a microphone and the processor includes voice-activated algorithms.

In some variations, the removable gas pathway module includes a tagging device capable of enabling or disabling the apparatus, and the processor includes an algorithm to disable the tagging device in response to a breath analysis.

In some variations, the apparatus includes a disposable sleeve to cover the apparatus. In some variations, the apparatus includes construction to allow sterilization such as autoclaving.

In some variations, the apparatus includes an outlet and a filter, wherein the gas is expelled to the ambient through the outlet and then the filter, and wherein the filter is non-permeable to at least one infectious disease.

In some variations, a method for screening for infectious disease includes: obtaining a breath sample from a subject, wherein the breath sample passes through a pathway in an instrument; analyzing, using a breath analyzer in the instrument, the breath sample for a presence and level of an analyte that correlates to the disease; determining the presence of the disease; removing the pathway from the instrument; and inserting a replacement pathway into the instrument.

In some variations, the analyte is CO and analyzing the breath sample for the presence and level of CO includes at least one of analyzing the rate of hemolysis, and analyzing a rate of hemolysis as an indicator of the infectious disease in the body, and analyzing a break-down of red blood cells based on the subject's response to the infectious disease.

In some variations, the infectious disease is Ebola.

In some variations, the method includes remotely commanding the instrument.

In some variations, the method includes modifying a treatment based on a measured level of the analyte.

In some variations, the method includes assessing the efficacy of a treatment based on the measured level of the analyte, and optimizing the treatment option by comparison of different treatments.

In some variations, obtaining the breath sample is performed automatically. In some variations, obtaining the breath sample includes discriminating multiple breaths to determine an appropriate breath for sampling.

In some variations, the analyte is a gaseous substance in the exhaled breath. In some variations, the analyte is a solid molecule.

In some variations, the sample is measured in real time during the test. In some variations, the sample is measured off-line.

The examples may describe for exemplary purposes an infectious disease breath analysis screening tool and method in which the analyte being measured is CO, for example when the infectious disease increases the rate of hemolysis. However this is exemplary and it should be understood that the methods and systems described herein apply to measuring other analyses present in the breath as a result of an infectious disease. Obtaining and measuring analyses from different sections of the breathing cycle are contemplated; from the complete exhalation cycle, or from the end-tidal section of exhalation, or from other sections of the breathing cycle, for example. Some variations include obtaining and measuring samples for either one breath, or multiple breaths, depending on the sample size requirements. Breath selection algorithms, in which an appropriate type of breath is defined and targeted for the analysis, the type of breath chosen based on the level and type of analyte in question, are also contemplated.

### BRIEF DESCRIPTION OF THE DRAWINGS

FIG. 1 describes a schematic overview of the breath analysis system, in accordance with an exemplary variation.

FIG. 2 describes a schematic diagram of a patient interface, in accordance with an exemplary variation.

FIG. 3a describes a schematic diagram of a pneumatic system in the sample collection mode, in accordance with an exemplary variation.

FIG. 3b describes a schematic diagram of the pneumatic system of FIG. 3a in the sample analysis mode, in accordance with an exemplary variation.

FIG. 4 shows a breath collection and measurement apparatus without a disposable module attached, in accordance with an exemplary variation.

FIG. 5 describes a disposable test module for the apparatus of FIG. 4, in accordance with an exemplary variation.

FIG. 6 describes the apparatus of FIG. 4 with the disposable self-contained test module attached, in accordance with an exemplary variation.

FIG. 7 describes the apparatus of FIG. 6 with a protective cover which completely shields the apparatus from exposure to infectious agents, in accordance with an exemplary variation.

FIG. 8 schematically describes a pneumatic system for collecting and analyzing breath samples, in accordance with an exemplary variation.

FIG. 9 describes a pneumatic system not capturing or analyzing a sample, in accordance with an exemplary variation.

FIG. 10 describes the apparatus of FIG. 9 in the sample collection state, in accordance with an exemplary variation.

FIG. 11 describes an alternative apparatus in which the sample is collected and available for off-line analysis, in accordance with an exemplary variation.

FIG. 12 describes a method for screening for infectious disease, in accordance with an exemplary variation.

### DETAILED DESCRIPTION

Described herein are methods for administering immune globulin and devices for use thereof. The methods may generally include measuring a patient's hemolysis levels and determining whether the patient is suitable for immune globulin treatment and/or determining whether immune globulin treatment should be continued. Because hemolysis can be a side-effect (systemic complications, leading to life threatening events such as acute renal failure and disseminated intravascular coagulation) of immune globulin treatment, the methods described herein may advantageously increase the success rate of immune globulin treatments by monitoring hemolysis before and during treatment.

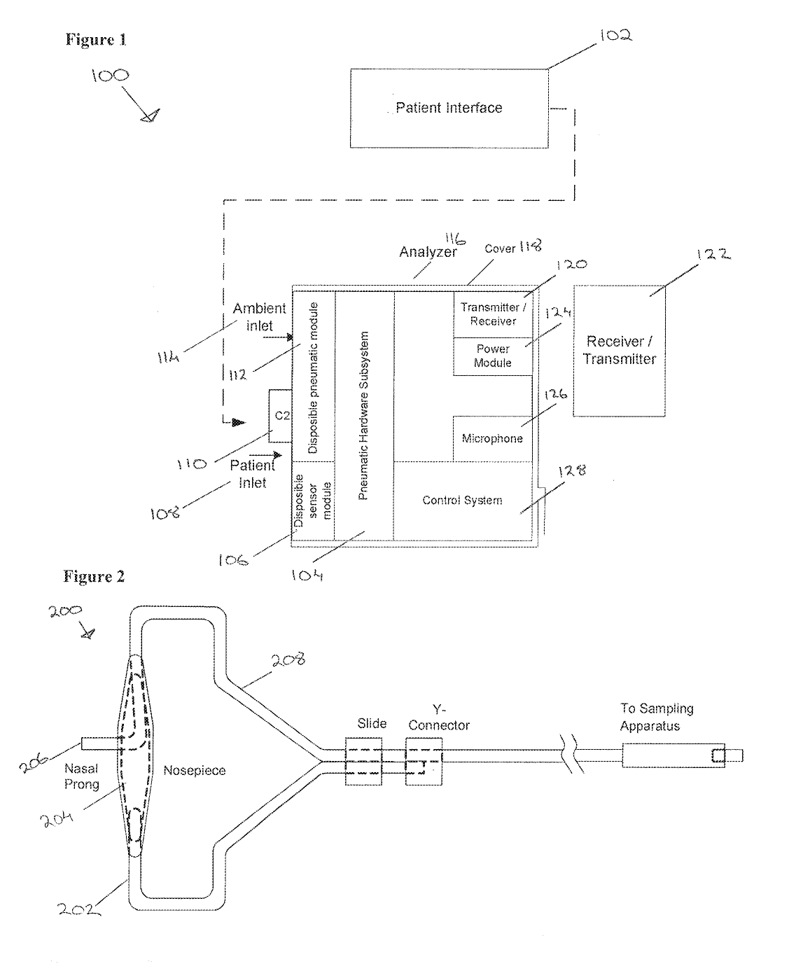

FIG. 1 depicts system 100, comprising a patient interface 102, an analyzer 116, a cover 118 for the analyzer, a removably attachable pneumatic module 112, a pneumatic hardware subsystem 104, a removably attachable analyte measurement sensor module 106, and optionally a physically separate control module receiver/transmitter 122, in accordance with a variation.

Separate control module 122 can be used to receive results from the instrument, for example if the instrument is in an isolation ward, or of an individual is using the instrument at home. The instrument may also be commanded to operate by voice command (using microphone 126), for example again if the instrument is in an isolation ward, or if the subject is separated from the health care provider supervising the test, or in the case of laboratory research in which the test subjects may not be easily accessible.

System 100 may also include a patient inlet 108, an ambit inlet 114, a transmitter/receiver 120 (for telecommunication with receiver/transmitter 122), a power module 124, and control system 128.

As will be seen in the subsequent descriptions, the removably attachable pneumatic module can analyze moving gas through a breath analysis instrument in a manner that does not come in contact with and therefore does not contaminate the hardware of the system.

FIG. 2 depicts patient interface 200, comprising a nosepiece 204 with a nasal prong 206, and sampling line 208 for connection to a sampling apparatus, in accordance with a variation. In some variations, interface 200 may be patient interface 102 displayed in FIG. 1. Patient interface 200 also includes a non sampling line 202 which may aid in positioning the patient interface on a patient's face.

The patient interface can be devoid of a filter that could trap the agent that is being looked for. In some variations, the patient interface can include a filter that traps the agent and is placed into the instrument for analysis.

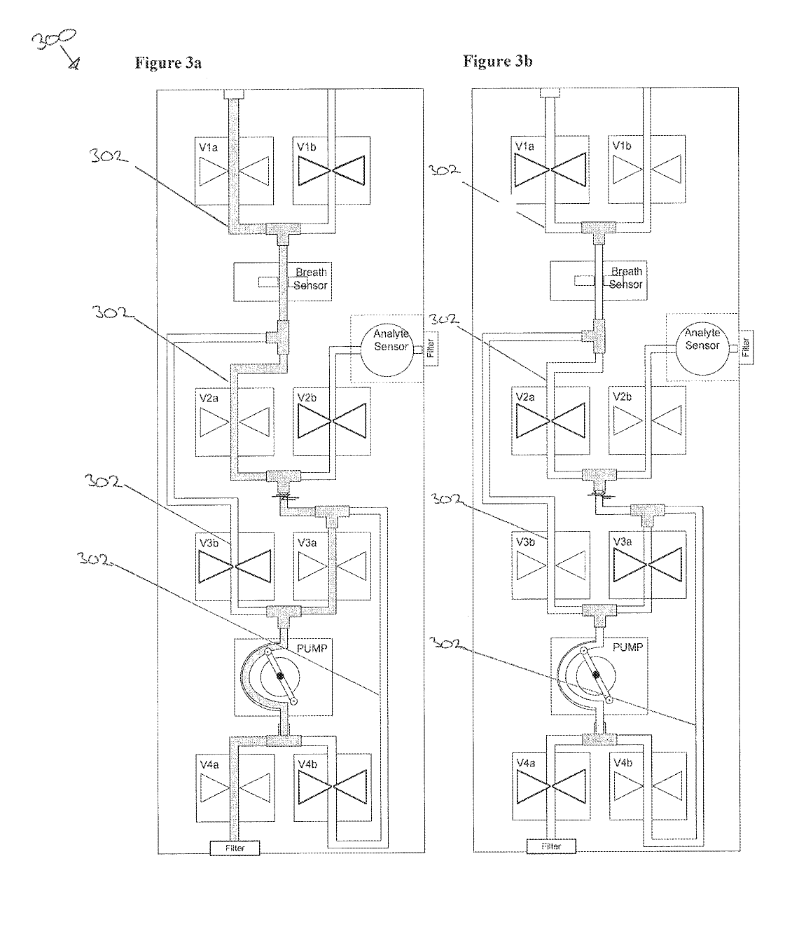

FIGS. 3a and 3b describe a combination 300 of a disposable pneumatic module 302 and pneumatic hardware subsystem (valving, pumps, etc. in FIGS. 3a and 3b), in accordance with a variation. Disposable pneumatic module 302 may correspond to disposable pneumatic module 112 in FIG. 1. Similarly, the pneumatic hardware subsystem in FIGS. 3a and 3b may correspond to pneumatic hardware subsystem 104 in FIG. 1. The analyte sensor in FIGS. 3a and 3b may correspond to disposable sensor module 106 in FIG. 1.

Removable pneumatic module 302 is shown snapped into the pneumatic hardware subsystem of the instrument. In FIG. 3a the pneumatics are shown in the breath sample collection mode. The hardware subsystem is arranged so that the flow of gas from the patient does not enter any of the pneumatic hardware components, such as the gas routing valves.

In some variations, the flow of gas does not pass through the analyte sensor. In other variations, the flow of gas does pass through the analyte sensor. The analyte sensor may be disposable.

Valves V1a, V1b, etc., may be pinching valves with mechanisms that pinch the gas flow tubing to route the travel of the gas. Therefore, the valve is never exposed to the possible contamination in the gas. In some variations, the valves are electromagnetic or otherwise do not contact the gas flowing in removable pneumatic module 302.

The breath sensor, if included, may comprise sensing elements that do not require physical contact with the gas or condensate. For example, the sensing elements can be optical or ultrasonic. The section tubing passing through the sensing elements may be of a different construction and properties that the balance of the tubing set, such as glass, polycarbonate, or other material that has the optical properties required for the sensor.

The pump may be of a type that propels gas flow without requiring physical contact with the gas, such as a peristaltic pump.

FIGS. 3a and 3b describe a variation for routing and segmenting gas from the patient as follows: The gas collection pathway (FIG. 3a) includes gas traveling through V1a, the Breath Sensor, through valve V2a, through a sample collection tube between V2a and V3a, through V3a, through the pump, and exhausting out through valve V4a and through a contaminant filter. During this phase, the opposing valves, V1b, V2b, etc., have the tubing pinched closed to divert the flow of gas through the other routes. Once a sample is collected between valve V2a and valve V3a, the system switches the valving to the “a” valves being pinched closed, and the “b” valves being switched open, as shown in FIG. 3b.

As shown in FIG. 3b, ambient air enters through valve V1b and through the Breathing Sensor, around valves V2a and V3b in a bypass tube and into valve V3b, through the Pump, through valve V4b, to the section between valve V3a and V2b where the breath sample has been located, and pushes the breath sample through valve V2b and to the Analyte Sensor and exhausting out through a contaminant filter. The contaminant filters at the exhaust vents are especially designed to trap any infectious agent from escaping into the environment and contaminating others. In some variations, these filters can be ultra-low porosity filters and can include an active element such as a UV light source, or heater tuned to annihilate or disintegrate the infectious agent within the instrument prior to discharge from the instrument.

In some variations, each pair of valves in FIGS. 3a and 3b is replaced with a three way pinching valve. The sections of tubing that are placed between the pinching elements of the valves and in the pump may be of a resilient material such as silicone or synthetic rubber. The tubing set can be arranged in an easy to insert module as will be described later. Alternate pneumatic pathways are also contemplated, and the pathways and valve controls described in FIGS. 3a and 3b are for exemplary purposes.

The system of FIGS. 3a and 3b may be advantageous in an application in which end-tidal gas is being measured for a specific infection, and in which a certain type of breath needs to be measured, rather than just any breath. The type of analytical sensor can be chemical, electrochemical, florescent, colorimetric, optical, or other types of sensing technology. In some applications it may be possible to combine the breath sensing function with the analyte sensing function, depending on the analyte in question, in which case either the breath sensor or analyte sensor can be omitted from the design.

In some variations, it may be sufficient to route the gas sample directly to the analyte sensor for measurement, rather than temporarily storing it in a collection area. In some variations, the instrument may be used to collect the sample only, and the sample is then inserted into another instrument. In some variations, the exhaust filter is modularly removable in a hermetically sealed module, to allow for sample archival and subsequent analysis. As illustrated, the exhaust filter is specially designed to trap the agent in question.

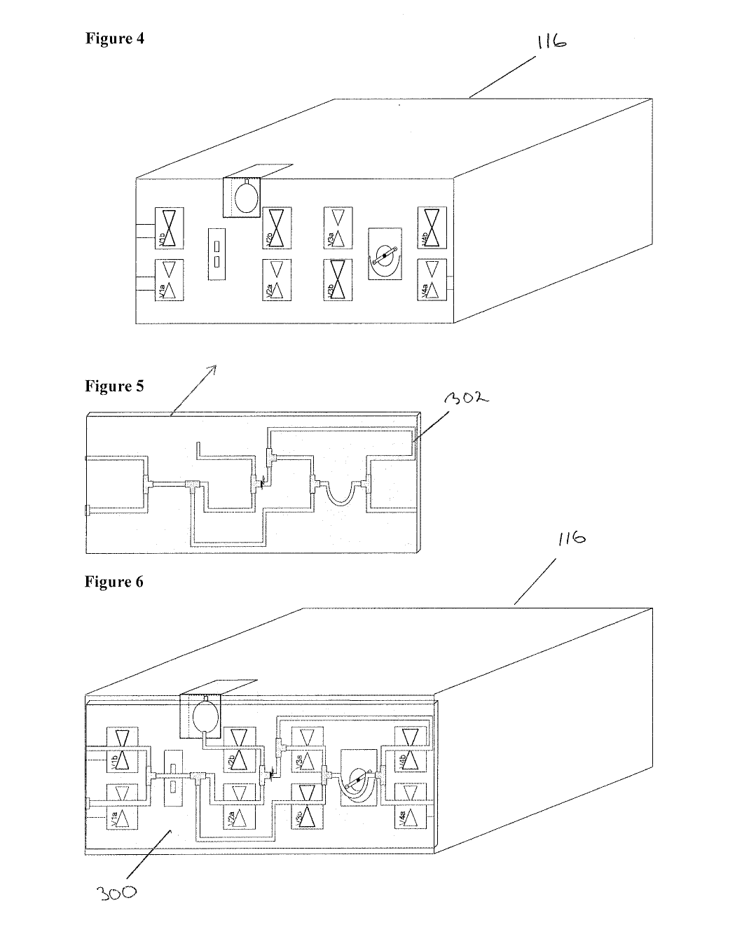

FIGS. 4-6 show a disposable removable pneumatic module 302 designed to be snapped into the pneumatic hardware subsystem of instrument 116, in accordance with an exemplary variation. The removable pneumatic module 302 may be made available on a film or plate so that the components of the assembly are precisely registered and the entire assembly can be easily, quickly and reliably snapped into the hardware side of the instrument with perfect placement of the disposable tubing relative to the hardware components.

FIG. 6 shows the disposable removable pneumatic kit attached to the instrument. The disposable kit may include a tagging feature and the instrument a tagging function, which together enable or disable the apparatus for performing a test. The tagging feature can allow the kit to be reused if the previous test is negative, or disable the apparatus if the previous test is positive. In this way, the tag can prevent a second use of the removable gas pathway module that may lead to an incorrect diagnosis due to cross contamination. In addition the tagging function can be used to make sure the kit is properly installed.

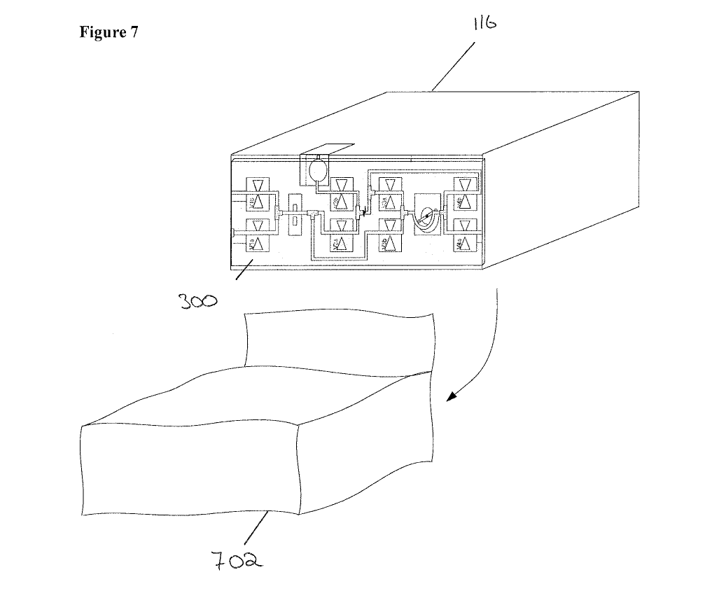

FIG. 7 describes a protective, transparent sleeve 702 in which the instrument may be placed to protect the surfaces of the instrument from exposure to contamination. After use, the sleeve can be easily removed and disposed of or sterilized.

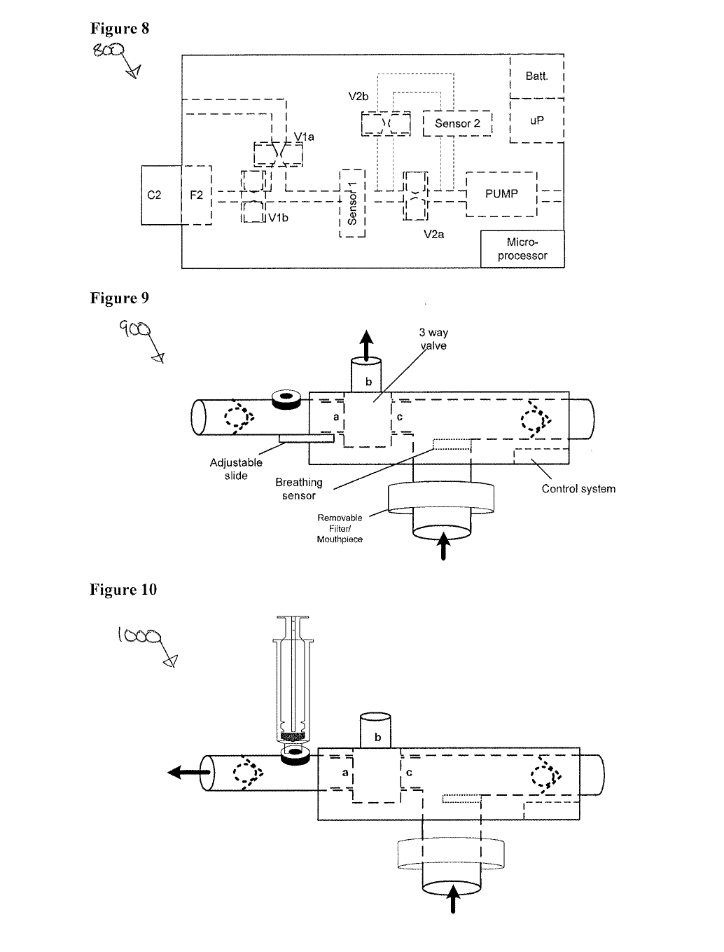

FIG. 8 describes apparatus 800 in which gas from the patient enters via the pinching valve V1a, through Sensor 1, and through a pump, with valves V1b and V2b pinched closed to divert flow through valves V1a and V2a. When the system determines a sample should be analyzed, the sample travels through valve V2b and through Sensor 2, then through the pump, with valves V1a and V2a pinched closed and valves V1b and V2b open. Sensor 1 is used to study the breathing status of the test subject, and Sensor 2 is used to measure the analyte. Sensor 1 can be a pressure transducer, flow sensor, CO2 sensor, or another type of sensor able to study the breathing status. Sensor 2 can be an electrochemical sensor, chemical sensor, electrical sensor, colorimetric sensor, optical sensor, illuminescence, bioluminescence sensor, chromatography sensor, or other types of sensors. In some variations, the same sensor is used to study the breathing status and measurement of the analyte.

FIGS. 9 and 10 describe a variation of an apparatus 900 where the patient interface and sample collection instrument are integrated into a small portable apparatus. The entire apparatus or substantial sections of the apparatus may be disposable or sterilize-able. The apparatus can be affixed to the test subject's airway, typically their nose or mouth, but also potentially coupled to the airway in other ways, and the subject breaths in and out through the apparatus. Normal breathing can take place, or breath maneuver commands can be given. The inspired gas enters the right side of the apparatus, and enters the subject through the removable filter, and expired gas exits the left side of the apparatus. Exhaled gas is trapped in the expiratory limb of the apparatus, and can be accessed through a sample collection port, for example with a syringe as shown in FIG. 10. Gaseous or non-gaseous analyses can be collected and analyzed accordingly.

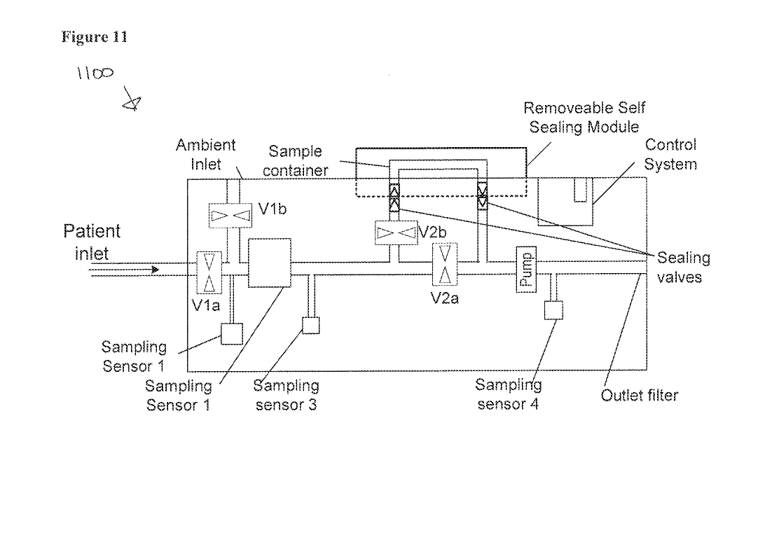

FIG. 11 describes apparatus 1100 in which case the sample measurement can be performed off line. The sample collection instrument is configured to store the sample, which can be preserved in a sealed container which is removable and adapted to attach to another instrument for analysis. The sample collection can be that of a single breath, the breath chosen based on breath selection criteria, or can be that of multiple breaths. Patient gas travels through valves V1a and V2a, and the selected sample from the patient is diverted through valve V2b, optionally with ambient air drawn in through valve V1b. Once the desired requisite sample is caught in the sample compartment, the gas flow changes back to path V1a-V2a.

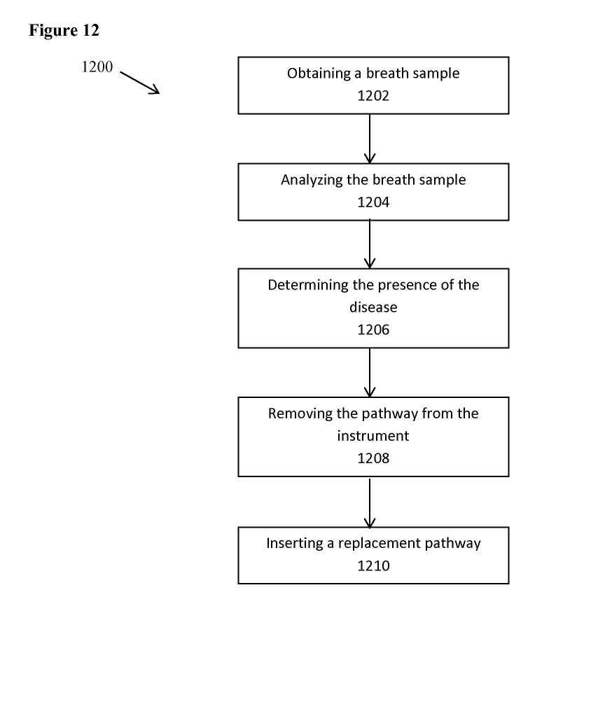

FIG. 12 describes a method 1200 for screening for infectious disease, in accordance with a variation. Method 1200 includes obtaining a breath sample from a subject 1202, wherein the breath sample passes through a pathway in an instrument. Method 1200 includes analyzing, using a breath analyzer in the instrument, the breath sample for a presence and level of an analyte that correlates to the disease 1204. Method 1200 includes determining the presence of the disease 1206. Method 1200 includes removing the pathway from the instrument 1208. Method 1200 includes inserting a replacement pathway into the instrument 1210.

In some variations, the analyte is CO and analyzing the breath sample for the presence and level of CO includes at least one of analyzing the rate of hemolysis, and analyzing a rate of hemolysis as an indicator of the infectious disease in the body, and analyzing a break-down of red blood cells based on the subject's response to the infectious disease.

In some variations, the infectious disease is Ebola.

In some variations, the method includes remotely commanding the instrument.

In some variations, the method includes modifying a treatment based on a measured level of the analyte.

In some variations, the method includes assessing the efficacy of a treatment based on the measured level of the analyte, and optimizing the treatment option by comparison of different treatments.

In some variations, obtaining the breath sample is performed automatically. In some variations, obtaining the breath sample includes discriminating multiple breaths to determine an appropriate breath for sampling.

In some variations, the analyte is a gaseous substance in the exhaled breath. In some variations, the analyte is a solid molecule.

In some variations, the sample is measured in real time during the test. In some variations, the sample is measured off-line.

In the foregoing descriptions of variations, it should be noted that it is also conceived that the sequences of operation described in the Figures can be combined in all possible permutations. In addition, while the examples describe a NO measurements they may apply to other gases and analyses. The examples provided throughout are illustrative of the principles of the systems and methods described herein, and that various modifications, alterations, and combinations can be made by those skilled in the art without departing from the scope and spirit of the invention. Any of the variations of the various breath measurement and sampling devices disclosed herein can include features described by any other breath measurement and sampling devices or combination of breath measurement and sampling devices herein. Accordingly, it is not intended that the invention be limited, except as by the appended claims. For all of the variations described above, the steps of the methods need not be performed sequentially.

1. A breath analysis apparatus comprising:
an inlet to obtain a flow of gas from a subject;
a breath detector to measure a breathing signal in the flow of gas;
a processor that determines an acceptable breath based on the breathing signal;
a modular subassembly containing the pathway for the flow of gas that is removable from the apparatus;
valves for controlling the flow of gas within the pathway, wherein the valves are fluidly disconnected from the flow of gas; and
an analyte composition sensor fluidly connected to the flow of gas.

2. The system of claim 1, wherein the valves are configured to allow the removable gas flow pathway to be snapped into the apparatus.

3. The system of claim 1, wherein the valves comprise pincher valves.

4. The system of claim 1, further comprising a peristaltic pump, and wherein the breath detector comprises an non-contact sensing element.

5. The system of claim 1, wherein the apparatus comprises a transmitter to transmit results to a physically remote receive for viewing by a user at a distance from the apparatus.

6. The system of claim 1, wherein the apparatus comprises a receiver, and wherein the apparatus is configured to analyze breaths in response to remote commands received through the receiver from a physically separate transmitter.

7. The system of claim 1, further comprising a microphone and wherein the processor comprises voice-activated algorithms.

8. The system of claim 1, wherein the removable gas pathway module comprises a tagging device capable of enabling or disabling the apparatus, and wherein the processor comprises an algorithm to disable the tagging device in response to a breath analysis.

9. The system of claim 1 further comprising a disposable sleeve to cover the apparatus.

10. The system of claim 1 further comprising construction to allow sterilization such as autoclaving.

11. The system of claim 1 further comprising an exhaust outlet and a filter, wherein the gas is expelled to the ambient through the outlet and then the filter, and wherein the filter is non-permeable to at least one infectious disease.

12. A method for screening for infectious disease, comprising:
obtaining a breath sample from a subject, wherein the breath sample passes through a pathway in an instrument;
analyzing, using a breath analyzer in the instrument, the breath sample for a presence and level of an analyte that correlates to the disease;
determining the presence of the disease;
removing the pathway from the instrument; and
inserting a replacement pathway into the instrument.

13. The method of claim 12 wherein the analyte is CO and wherein analyzing the breath sample for the presence and level of CO comprises at least one of analyzing the rate of hemolysis, and analyzing a rate of hemolysis as an indicator of the infectious disease in the body, and analyzing a break-down of red blood cells based on the subject's response to the infectious disease.

14. The method of claim 12, wherein the infectious disease is Ebola.

15. The method of claim 12 further comprising remotely commanding the instrument.

16. The method of claim 12 further comprising modifying a treatment based on a measured level of the analyte.

17. The method of claim 12 further comprising
assessing the efficacy of a treatment based on the measured level of the analyte, and
optimizing the treatment option by comparison of different treatments.

18. The method of claim 12, wherein obtaining the breath sample is performed automatically.

19. The method of claim 12, wherein obtaining the breath sample further comprises discriminating multiple breaths to determine an appropriate breath for sampling.

20. The method of claim 12, wherein the analyte is a gaseous substance in the exhaled breath.

21. The method of claim 12, wherein the analyte is a solid molecule.

22. The method of claim 12, wherein the sample is measured in real time during the test.

23. The method of claim 12, wherein the sample is measured off-line.
